(19) United States
(12) Patent Application Publication
(10) Pub. No.: US 2025/0144324 A1
(43) Pub. Date: May 8, 2025

(54) CNS INHALER AND OPERATING METHOD THEREOF

(71) Applicant: NEUCEN BIOMED CO., LTD., Taipei City (TW)

(72) Inventor: MING-FU CHIANG, Taipei City (TW)

(21) Appl. No.: 18/835,012

(22) PCT Filed: Dec. 15, 2022
(86) PCT No.: PCT/CN2022/139244

§ 371 Date: Jul. 31, 2024

(30) Foreign Application Priority Data
Jun. 1, 2022 (CN) 202210617307.0

(51) Int. Cl. A61M 15/00; A61M 11/00

(52) U.S. Cl. CPC A61M 15/0001; A61M 11/003; A61M 11/005; A61M 2202/0007; A61M 2202/04; A61M 2205/0233; A61M 2205/025; A61M 2205/12; A61M 2205/3386; A61M 2205/50

(57) ABSTRACT

The present disclosure discloses a CNS inhaler and an operating method thereof. The inhaler is constructed by a disposable module and a grip. The grip is detachably connected to the disposable module. The disposable module comprises a pre-stored medicine container, a mesh, an oscillator, a holder and an adaptor. The grip comprises a control element and a trigger element. The disposable module of the present disclosure supplies medicine in a preset mode for the human CNS. After the supply has been finished, the disposable module may just be separated from the grip and abandoned.

### CROSS-REFERENCE TO RELATED APPLICATION

This application claims priority to Chinese Patent Application No. 202210617307.0, filed on Jun. 1, 2022, which is incorporated herein by reference in its entirety.

### TECHNICAL FIELD

The present disclosure relates to a central nervous system (CNS) inhaler and an operating method thereof. Specifically, the CNS inhaler and the operating method per se are used for delivering medicine to the central nervous system of human body.

### BACKGROUND

In the technical field of respiratory therapy, an inhaler is a common device. Specifically, several indications may use inhalers for medicine delivery such as chronic obstructive pulmonary disease (COPD) or asthma, etc., which are the indications use the inhalers.

However, the technology develops and the type of indications which may use inhalers is no longer limited by the cardiovascular or respiratory related diseases. In fact, nano-drugs related to a cancer treatment may be delivered to the human body via inhalation.

In the use of the inhalation medicine, it is recommended to directly target the nasal pharynx for medicine delivery, or even to directly target the central nervous system across the blood-brain barrier (BBB) then deliver.

That is, precise instrument such as the inhaler with medicine delivery function are created respectively, but these inhalers are still disadvantageous for using. In other words, the commercial precise inhaler is required to inject the medicine via an on/off inlet. The reusable characteristic may challenge the doctors and nurses in accuracy of dosing, and the risk of contamination of the external environment is relatively higher.

### SUMMARY

To solve the problems mentioned in the background of related arts, the present disclosure provides a CNS inhaler and operating method thereof. The inhaler is mainly constructed by a disposable module and a grid. The grid is connected to the disposable module.

The disposable module comprises a pre-stored medicine container, a mesh, an oscillator, a holder and an adaptor. The mesh and the pre-stored medicine container are connected to each other. The oscillator and the mesh are connected, too. On the other hand, the holder covers the mesh, and the adaptor is connected to the holder.

Furthermore, the grid comprises a controller and a trigger. The controller detachably connected to the oscillator, and the trigger is connected to the controller.

As the above-mentioned CNS inhaler, the present disclosure further provides an operating method of the CNS inhaler. In step (A), the abovementioned CNS inhaler is provided. In step (B), the disposable module is selected via a type of the pre-stored medicine container.

Further in step (C), the disposable module is connected with the grid, and the oscillator is connected to the controller. Thereinafter, the step (D) states that the trigger triggers the controller to control the oscillator oscillating the mesh via a frequency.

After the step (D) has been finished, the step (E) states that the mesh pressures the medicine stored in pre-stored medicine container and vaporizing the medicine to turn into a medicine moisture. At last, in the step (F), after the medicine stored in pre-stored medicine container are vaporized and turned into the medicine moisture, the disposable module and the grid are detached, and the disposable module is abandoned.

Embodiments of the disclosure are illustrated by way of example, and not by way of limitation, in the figures of the accompanying drawings in which like reference numerals refer to similar elements.

### BRIEF DESCRIPTION OF THE DRAWINGS

FIG. 1 is a schematic structure diagram of a CNS inhaler of some embodiments of the present disclosure.

FIG. 2 is another schematic structure diagram of a CNS inhaler of some embodiments of the present disclosure.

FIG. 3 is a flow chart of a CNS inhaler operating method of some embodiments of the present disclosure.

### DETAILED DESCRIPTION

In order to make the description of the present disclosure more detailed and complete, the following description provides an illustrative description for the implementation and specific embodiments of the present disclosure.

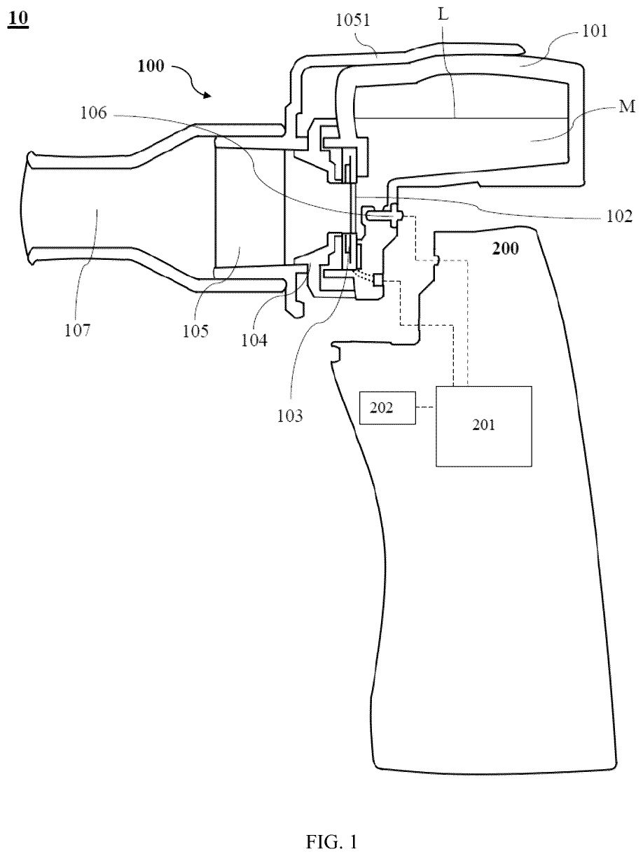

Please refer to FIG. 1. FIG. 1 is a schematic structure diagram of a CNS inhaler of some embodiments of the present disclosure. As shown in FIG. 1, some embodiments provide a CNS inhaler 10. The CNS inhaler 10 is mainly constructed by a disposable module 100 and a grid 200. The grid 200 is detachably connected to the disposable module 100.

In some embodiments, the shape of the grid 200 may be cylindrical, gun handle shape or the other shell with a shape which is convenient for the users' hand to grip. Furthermore, the grid 200 may comprise all essential units which is related to electromechanical control such as battery or circuit board, the present disclosure is not limited thereto.

On the other hand, the disposable module 100 is detachably connected to the grid 200 via a simple mechanical structure. Specifically, the simple mechanical structure may be the rail structure, male and female connecting structure or the magnetic connecting structure while the disposable module 100 and grid 200 both have the electromagnetic protection structure. The present disclosure is not limited thereto, too.

In addition, the disposable module 100 of some embodiments comprises a pre-stored medicine container 101, a mesh 102, an oscillator 103, a holder 104 and an adaptor 105. The mesh 102 is connected to the pre-stored medicine container 101, and the oscillator 103 is connected to the mesh 102. Moreover, the holder 104 covers on the mesh 102, and the adaptor 105 is connected to the holder 104.

The material of the pre-stored medicine container 101 of the present embodiment may be selected form the materials which is safe for storing medicines such as glass, shading glass or plastics etc. In other words, the pre-stored medicine container 101 controls the dosage of medicine which is used for treating different indications via pre-injecting the medicine with liquid level L. Therefore, the pre-stored medicine container 101 can not only avoid the dosage error and the risk of contamination which is caused by repeated use of medical staff, the pre-stored medicine container 101 is helpful for isolating and preserving the medicine from the external environment after the medicine has been pre-stored.

The material of the mesh 102 which is solely connected to the pre-stored medicine container 101 in the present embodiment is stainless steel, plastic or platinum. The material of the mesh 102 is determined by the type of medicine stored in the pre-stored medicine container 101. The material of the mesh 102 is preferably selected form the material which will not react with, contaminate, or cause denaturation of the medicine. Otherwise, the edge of mesh 102 is required to seal with the pre-stored medicine container 101.

The CNS inhaler 10 of the present embodiment is used for delivering the medicine for a central nervous system. The medicine stored in the pre-stored medicine container 101 may be temozolomide, dopamine and botanical extract. The indications of the temozolomide comprise the recurrent malignant glioma, and the indications of the dopamine comprise shock failure or heart failure. The botanical extract may treat cranial nerve repair or increase synaptic extension, the present disclosure is not limited thereto.

In order to the CNS inhaler 10 of some embodiments is used for delivering medicine for central nervous system. The pore diameter of mesh 102 in this embodiment ranges form 0.3-15 μm. Therefore, the particle size of medicine moisture vaporized by mesh 102 which is oscillated by the oscillator 103 ranges from 10-15 μm. Specifically, the preferred particle size of medicine moisture is 10 um. The preferred particle size is determined by the delivery path of the present embodiment. The delivery path of the present embodiment is that the vaporized medicine moisture is inhaled through the nasal cavity and is administered through the olfactory nerve channel (Crista Galli) into the central nervous system, such as the frontal lobe of the brain.

The oscillator 103 of some embodiments is an ultrasonic oscillator. Considering to the size limitation of disposable module 100, the oscillator 103 may use the piezoelectric membrane to convert the electrical energy into mechanical energy for oscillation, therefore to realize the ultrasonic oscillator. Furthermore, the frequency of oscillator 103 is set from 110 to 130 kHz. The preferred frequency is 120 KHz. The oscillator 103 may transfer the resonant mechanical energy to the mesh 102, vaporizing the medicine stored in the pre-stored medicine container 101 and releasing the medicine moisture from the pores of mesh 102.

The CNS inhaler 10 is mainly used for the inhalation of users. Hence, the holder 104 with the ring structure of the present embodiment stops, engages with, or retains the mesh 102, avoiding the asphyxia caused by sucking the thin and light mesh 102 during the inhalation vacuum of user. Thus, the connection between the oscillator 103 and mesh 102 of the present embodiment may prevent sucking of the mesh 102, further makes the holder 104 become the second security of the mesh 102.

On the other hand, the adaptor 105 of the present embodiment further detachably connects to a mask (not shown in drawings) or inhalation tube 107. In fact, the adaptor 105 of the present embodiment is used for the medical staff or users determining different ways to deliver the medicine via different delivery instruments.

In some embodiments, the adaptor 105 is not only configured on the holder 104, but also further comprises a shading portion 1051. The shading portion 1051 of the present embodiment is designed in one piece, therefore to be the extension top cap of the adaptor 105.

The shading portion 1051 shades/covers at least one part of pre-stored medicine container 101. The shading portion 1051 not only prevents/decreases the potential risk of denature of the medicine stored in pre-stored medicine container 101 due to the light, the shading portion 1051 also provides the additional protection for pre-stored medicine container 101, avoiding breaking the pre-stored medicine container 101 via collision thereon. The present disclosure is not limited thereto.

Furthermore, a liquid level sensor 106 is configured on/inside the pre-stored medicine container 101 of the present embodiment, and the liquid level sensor 106 is connected to the controller 201. Specifically, the liquid level sensor 106 is used for checking whether the medicine stored in the pre-stored medicine container 101 has been completely released or not. When the medicine stored in the pre-stored medicine container 101 has been released, the liquid level sensor 106 may sent the signal to the controller 201 and the controller 201 may notify the user that the medicine has been delivered.

Thereafter, the grid 200 of FIG. 1 comprises controller 201, trigger 202 or alternatively further comprises a memory 203. The controller 201 is detachably connected to the oscillator 103 and liquid level sensor 106, or the controller 201 may alternatively connects to the memory 203. The trigger 202 is connected to the controller 201. In some embodiments, the controller 201 is realized by a microprocessor.

In fact, the controller 201 may further comprise or alternatively select as a chip with control function such as the microcontroller unit (MCU), the present disclosure is not limited thereto. On the other hand, the trigger 202 may be mechanical buttons, touch panels or resistive and capacitive sensing devices etc., the present disclosure is not limited thereto, too.

The trigger 202 may be designed as an optical fingerprint sensor or a capacitive fingerprint sensor which have fingerprint recognition functions, allowing the authorized user or medical staff to release the medicine.

Hence, when controller 201 receives the signal activated by trigger 202, the oscillator 103 turns on and the operation of vaporization will be executed. The liquid level sensor 106 monitors the liquid level L of the medicine stored in the pre-stored medicine container 101 in real time. Thereafter, the controller 201 may check, control, or stop the signal from the trigger 202.

For instance, if the trigger 202 is a mechanical bottom, the analog signal generated by the pressure of trigger 202 may be conversed to related amount of digital signal and send to the controller 201. Therefore, the controller 201 may determine the current value and control the oscillator 103, such as the duration time or frequency oscillator 103, the present disclosure is not limited thereto.

To maintain the electromechanical control function between the controller 201 and trigger 202, the grid 200 may comprises the unit(s) which is helpful for the operation of the controller 201 and the trigger 202, such as rechargeable batteries, circuit protection components, wired/wireless communication modules etc., the present disclosure is not limited thereto.

The rechargeable batteries may supply the energy for the oscillator 103 which is connected to the controller 201 and the controller 201 under the protection of battery management system (BMS). The rechargeable batteries are selected form lithium batteries, lithium polymer batteries, lithium iron phosphate batteries or aluminum batteries, etc. Otherwise, the rechargeable batteries may be alternatively switched to the wired power such as the utility power. The utility power may be converted into the voltage reduced DC power and supply the energy to the controller 201 and the oscillator 103, the present disclosure is not limited thereto.

When the grid 200 needs the other reference data or the recordable data of the disposable module 100 which has been operated by the grid 200, the controller 201 may alternatively connects to an additional memory 203 of the grid 200. The aforementioned memory 203 may be a flash memory, a solid-state disk (SSD) or etc. The present disclosure is not limited thereto.

Specifically, the trigger 202 recited in FIG. 1 may directly output the digital signal (the trigger 202 comprises analog/digital converter). Therefore, in the embodiment of FIG. 1, the memory 203 comprises a trigger activity detecting program. The controller 201 may executes the trigger activity detecting program saved in the memory 203. Hence, the controller 201 may directly reads the electrical signal which is output via the trigger 202. The trigger activity detecting program may read the triggering times, period or estimated drug flow rate or the other parameters of the trigger 202, the present disclosure is not limited thereto.

When the grid 200 needs to communicates with the other terminals, the controller 201 further connects to a wireless communication module. The wireless communication module of some embodiments comprises but not limited to a near-field communication (NFC), a radio frequency identification (RFID), a WiFi™, a 4G™, a 5G™, a Bluetooth™ and the necessary hardware per se.

In other words, when the controller 201 is matched with the wireless communication module, the tags or chips (i.e., NFC chip or RFID tag) configured inside the disposable module 100 may rapidly send the information of medicine stored in the disposable module 100 to the controller 201 via the wireless communication module. The information of medicine stored in the disposable module 100 may comprises type, dosage, volume, concentration, conservation period, date of manufacture, manufacturing place, supplier, and applicable indications, etc. Therefore, the controller 201 may send the information of medicine stored in the pre-stored medicine container 101 of disposable module 100 such as type, dosage, volume, concentration, conservation period, date of manufacture, manufacturing place, supplier, and applicable indications, etc. to at least one terminal device via the same or the other wireless communication module which is connected to the controller 201.

The terminal devices may be a smartphone, a tablet, a laptop or a PC, the present disclosure is not limited thereto. After the terminal device receives the information of the medicine stored in the pre-stored medicine container 101 of the disposable module 100 such as type, dosage, volume, concentration, conservation period, date of manufacture, manufacturing place, supplier, and applicable indications, the information may be directly displayed on the screen of the terminal devices, providing the user or the medical staff to immediately, correctly and quickly check that the current disposable module 100 which is connected to the grid 200 is the correct one or not.

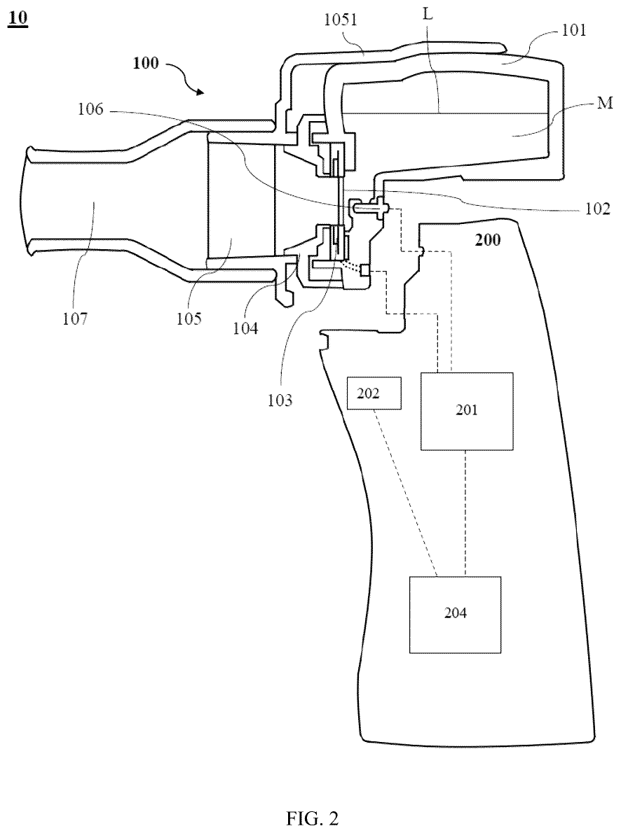

Please refer to FIG. 2. FIG. 2 is another schematic structure diagram of the CNS inhaler of some embodiments of the present disclosure. As shown in FIG. 2, the difference between the embodiments of FIG. 2 and FIG. 1 is that the signal output by the trigger 202 of FIG. 2 is analog signal. Therefore, a trigger activity detecting unit 204 is further configured between the controller 201 and the trigger 202.

In some embodiments, the trigger activity detecting unit 204 is constructed by an analog/digital converter and a memory. The analog/digital converter of the trigger activity detecting unit 204 may converts the analog signal transmitted from the trigger 202 into the digital signal. Simultaneously, the controller 201 may execute the trigger activity detecting program which is saved in the memory of trigger activity detecting unit 204. The digital signal such as triggering times, period or estimated drug flow rate . . . etc. which are converted by the analog/digital converter of trigger activity detecting unit 204 may be transmitted to the controller 201.

Hence, the controller 201 may execute the trigger activity detecting program which is saved in the memory of trigger activity detecting unit 204, detecting and recording the parameters such as triggering times, period or estimated drug flow rate of the trigger 202.

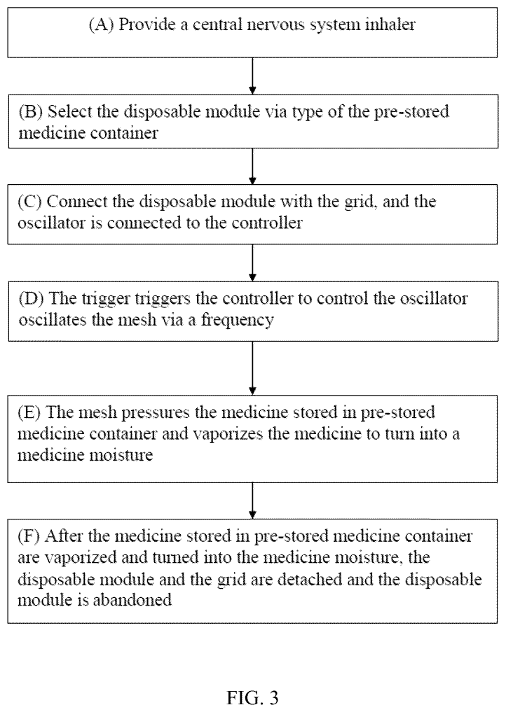

Please refer to FIG. 1, FIG. 2 and FIG. 3 simultaneously. The FIG. 3 is a flow chart of CNS inhaler operating method of a preferable embodiment of the present disclosure. As shown in FIG. 3, the operating method of CNS inhaler as illustrated in FIG. 3 is basically refer to the operating method of the CNS inhaler 10 illustrated in FIG. 1 or FIG. 2.

Specifically, the step (A) of the present embodiment is to provide the CNS inhaler 10 as described in FIG. 1. The step (B) is to select the disposable module 100 via the type of the pre-stored medicine container 101. The type of the pre-stored medicine container 101 means that the indications of the liquid medicine stored in the pre-stored medicine container 101.

As the description of the embodiment of FIG. 1, the type of the pre-stored medicine container 101 may be check by at least one terminal device for the user(s) or medical staff via the tag or chip (i.e., NFC chip or RFID tag) which is configured inside the disposable module 100.

Thereinafter, the step (C) is to connect the disposable module 100 and the grid 200, establishing the connection between the oscillator 103 and the controller 201. As the description above, the connection between the disposable module 100 and the grid 200 may be a simple mechanical structure such as rail structure, male and female connecting structure or the magnetic connecting structure while the disposable module 100 and the grid 200 both have the electromagnetic protection structure, the present disclosure is not limited thereto.

Moreover, in the step (C), after the disposable module 100 and the grid 200 are connected, the adaptor 105 alternatively connects to a mask (not shown in drawings) or the inhalation tube 107 of some embodiments. The mask or the inhalation tube 107 of some embodiments is used for fitting and adapting the face of the user/patient then execute step (D).

The step (D) of some embodiments is to use the trigger triggering the controller to control the oscillator oscillating the mesh via a frequency. As the description above, the trigger 202 may respond the command of the user or medical staff to controller 201, controlling the oscillation frequency of the oscillator 103. In some embodiments, the frequency ranges from 110 to 130 kHz, and 120 kHz is preferred.

After the step (D), the step (E) is that the mesh pressures the medicine stored in the pre-stored medicine container and vaporizes the medicine to turn into a medicine moisture. In the step (E), the mesh 102 is oscillated and pressured by the oscillator 103 via an ultrasonic oscillation, a pressure may pressure the liquid medicine stored in the pre-stored medicine container 101. In order to the structure of mesh 102 is porous structure, the area with pores may generates the exit of pressure. Therefore, the liquid medicine stored in the pre-stored medicine container 101 will be pressured and vaporized to turn into particles, forming the medicine moisture.

At last, the step (F) is that the medicine stored in the pre-stored medicine container are vaporized and turned into the medicine moisture, the disposable module and the grid are detached, and the disposable module is abandoned. In the step (F), the controller 201 may sense whether the medicine stored in the pre-stored medicine container 101 has been completely vaporized and turned into the medicine moisture via the liquid level sensor 106. After the required dosage of the medicine stored in the pre-stored medicine container 101 of the disposable module 100 has been completely released, the disposable module 100 can be abandoned, preventing the contamination caused by second use.

The above-mentioned descriptions are only preferred embodiments of the present disclosure and are not intended to limit the scope of implementation of the present disclosure. Therefore, all the shapes, structures, features and spirits described in the scope of the patent application of the present disclosure shall be regard as equivalent to the changes and modifications per se, and be included in the scope of the patent application of the present disclosure.

What is claimed is:

1. A central nervous system inhaler, comprising:
a disposable module, comprising:
a pre-stored medicine container;
a mesh, connected to the pre-stored medicine container;
an oscillator, connected to the mesh;
a holder, covered on the mesh;
a adaptor, connected to the holder;
a grid, detachably connected to the disposable module; wherein the grid comprises:
a controller, detachably connected to the oscillator; and
a trigger, connected to the controller.

2. The central nervous system inhaler according to claim 1, wherein a liquid level sensor is further configured on the pre-stored medicine container, and the liquid level sensor is connected to the controller.

3. The central nervous system inhaler according to claim 1, wherein pore diameter of the mesh ranges between 0.3-15 μm.

4. The central nervous system inhaler according to claim 1, wherein the mesh is oscillated by the oscillator, and a medicine moisture which has a particle diameter which ranges between 10-15 μm is vaporized by the mesh.

5. The central nervous system inhaler according to claim 1, wherein material of the mesh is stainless steel, plastic or platinum.

6. The central nervous system inhaler according to claim 1, wherein the oscillator is an ultrasonic oscillator.

7. The central nervous system inhaler according to claim 1, wherein a frequency of the oscillator ranges from 110 to 130 kHz.

8. The central nervous system inhaler according to claim 1, wherein the controller is a microprocessor.

9. The central nervous system inhaler according to claim 1, wherein the adaptor further detachably connected to a mask or an inhalation tube.

10. The central nervous system inhaler according to claim 1, wherein the adaptor further comprises a shading portion, and the shading portion shades at least one part of the pre-stored medicine container.

11. The central nervous system inhaler according to claim 1, wherein the medicine stored in the pre-stored medicine container is temozolomide or dopamine.

12. The central nervous system inhaler according to claim 1, wherein a trigger activity detecting unit is further configured between the controller and the trigger.

13. The central nervous system inhaler according to claim 1, wherein the controller further connects to a memory, and the controller executes a trigger activity detecting program which is saved in the memory.

14. An operating method of a central nervous system inhaler, comprising:
(A) providing the central nervous system inhaler according to claim 1;
(B) selecting the disposable module via type of the pre-stored medicine container;
(C) connecting the disposable module with the grid, and the oscillator is connected to the controller;
(D) the trigger triggering the controller to control the oscillator oscillates the mesh via a frequency;
(E) the mesh pressuring the medicine stored in pre-stored medicine container and vaporizing the medicine to turn into a medicine moisture; and
(F) after the medicine stored in pre-stored medicine container are vaporized and turned into the medicine moisture, the disposable module and the grid are detached and the disposable module is abandoned.

15. The operating method of the central nervous system inhaler according to claim 14, wherein a liquid level sensor is further configured on the pre-stored medicine container, and the liquid level sensor is connected to the controller.

16. The operating method of the central nervous system inhaler according to claim 15, wherein in step (F), the liquid level sensor checks whether the medicine stored in the pre-stored medicine container has been completely vaporized and turned into the medicine moisture or not.

17. The operating method of the central nervous system inhaler according to claim 14, wherein in step (D), a frequency of the oscillator ranges from 110 to 130 kHz.

18. The operating method of the central nervous system inhaler according to claim 14, wherein in step (C), the adaptor detachably connected to a mask or an inhalation tube.
